(19) United States
(12) Patent Application Publication
(10) Pub. No.: US 2021/0386815 A1
(43) Pub. Date: Dec. 16, 2021

(54) Nutraceuticals for Reducing Myeloid Suppressor Cells

(71) Applicant: Theraputic Solutions International, Inc., Oceanside, CA (US)

(72) Inventors: Thomas E. Ichim, Oceanside, CA (US); Timothy G. Dixon, Oceanside, CA (US); James Veltmeyer, Oceanside, CA (US)

(73) Assignee: Therapeutic Solutions International, Inc., Oceanside, CA (US)

(21) Appl. No.: 16/898,472

(22) Filed: Jun. 11, 2020

(51) Int. Cl. A61K 36/82; A61K 36/45; A61K 36/31; A61P 35/04

(52) U.S. Cl. CPC A61K 36/82; A61P 35/04; A61K 36/31; A61K 36/45

(57) ABSTRACT

Disclosed are compositions of matter, treatments and protocols useful for reduction of number and/or activity of myeloid suppressor cells (MSC). In some embodiments the invention teaches the administration of a therapeutic combination of ingredients comprising of pterostilbene, Nigella sativa, sulforaphane, and epigallocatechin-3-gallate (EGCG) to a mammal at possessing an increased number and/or activity of said MSC in which reduction of number and/or activity is desired. In another embodiment, the invention teaches administration of said therapeutic combination to a mammal infected with viral and/or bacterial infections and/or neoplasia. In some embodiments dosage of said therapeutic combination is based on inflammatory and/or immunological parameters observed in patients.

### FIELD OF THE INVENTION

The invention pertains to the field of cancer immunotherapy, more specifically, the invention pertains to the use of natural compounds to elicit immune modulation, more specifically, the invention pertains to the field of reducing myeloid suppressor cells through administration of natural compounds.

### BACKGROUND OF THE INVENTION

Host immunity to cancers has been extensively documented both in animal models and humans going back to the days of William Coley. In fact, there is strong evidence that the immune surveillance plays a critical role in limiting tumor outgrowth in the early stages of tumorigenesis, for example, this is way transplant recipients, who are under constant immune suppression have higher rates of cancer. However, the ability to prime tumor-specific T-cells and sustain an immune response that imparts a measurable clinical benefit, is limited in the setting of an established tumor burden. Taken together, these findings suggest numerous requirements for effective immunotherapy. Tumor-specific T cells must not only possess a sizeable precursor frequency and reach sufficient numbers following activation, but they must also be able to traffic to the tumor site and effectively kill their targets in situ.

Growing tumors are able to modify their microenvironment and render it more immunosuppressive. Such intratumoral changes include altering the cytokine milieu, changing the extracellular matrix, and recruiting immune cells with a suppressive function. In mice, the CD11b.sup.+/Gr1.sup.+ MSCs represent one population of cells within the tumor microenvironment responsible for the immunosuppression accompanying tumor growth. Their elimination in tumor-bearing hosts restores CD8.sup.+ T cell responsiveness. This observation points to a reversible process and supports the hypothesis that strategies aimed at the pharmacologic inhibition of these pathways can be effective in restoring immune responsiveness. L-Arginine metabolism is a key pathway used by MSCs to blunt the anti-tumor response both in mice and humans. Arg1 and NOS2, the main enzymes that catabolize L-arginine, can, in fact, work either alone or synergistically in restrain T-cells response. Through an understanding of these critical suppressive pathways, it is possible to determine whether selective immunopharmacologic targeting can augment anti-tumor immunity. Nitroaspirin derivatives were recently shown to down-regulate NOS2 expression in tumor associated MSCs and to abrogate MSC-mediated immune-suppression in vivo but the mechanisms of these effects were not defined. To date there are not practical means of inducing a reduction in MSC numbers that can be rapidly translated.

### SUMMARY

Preferred embodiments are directed to methods of inhibiting number and/or activity of myeloid suppressor cells comprising administration of a therapeutic combination comprising of: a) Green Tea and/or extract thereof; b) Blueberry and/or extract thereof; c) Nigella sativa and/or extract thereof; and d) broccoli and/or extract thereof.

Further methods include embodiments wherein said green tea extract is epigallocatechin-3-gallate or an analogue thereof.

Further methods include embodiments wherein said blueberry extract is pterostilebene or an analogue thereof.

Further methods include embodiments wherein said Nigella sativa extract is thymoquinone or an analogue thereof.

Further methods include embodiments wherein said broccoli extract is sulforaphane or an analogue thereof.

Further methods include embodiments wherein said therapeutic combination is administered at a dosage and frequency sufficient to inhibit MSC number and/or activity.

Further methods include embodiments wherein inhibition of MSC number and/or activity in the host is associated with enhancement of natural killer cell activity.

Further methods include embodiments wherein said natural killer cell activity is quantified by ability to lyse a virally infected cell.

Further methods include embodiments wherein said natural killer cell activity is quantified by ability to lyse K562 cells.

Further methods include embodiments wherein said natural killer cell activity is quantified by ability to lyse YAC-1 cells.

Further methods include embodiments wherein inhibition of MSC number and/or activity in the host is associated with enhancement of interferon production.

Further methods include embodiments wherein inhibition of MSC number and/or activity in the host is accomplished by enhancement of T cell activation.

Further methods include embodiments wherein said T cell activation is induction of T helper cell 1 activity.

Further methods include embodiments wherein said T helper cell 1 activity comprises production of interferon gamma.

Further methods include embodiments wherein said T cell activation is induction of T cytotoxic cell activity.

Further methods include embodiments wherein said therapeutic combination is administered at a dosage and frequency sufficient to suppress growth of a tumor.

Further methods include embodiments wherein said tumor growth in the host is associated with suppression of cancer angiogenesis.

Further methods include embodiments wherein said cancer angiogenesis comprises of: a) endothelial cell detachment; b) migration towards a chemotactic gradient; and c) tube formation.

Further methods include embodiments wherein said therapeutic mixture decreases tumor associated fibroblasts.

Further methods include embodiments wherein said tumor associated fibroblasts secrete immune suppressive factors.

Further methods include embodiments wherein said immune suppressive factors are interleukin-4.

Further methods include embodiments wherein said immune suppressive factors are interleukin-10.

Further methods include embodiments wherein said immune suppressive factors are interleukin-13.

Further methods include embodiments wherein said immune suppressive factors are interleukin-20.

Further methods include embodiments wherein said immune suppressive factors are TGF-beta.

Further methods include embodiments wherein said immune suppressive factors are HGF.

Further methods include embodiments wherein said immune suppressive factors are VEGF.

Further methods include embodiments wherein said immune suppressive factors are PDGF.

Further methods include embodiments wherein said immune suppressive factors are FGF-1.

Further methods include embodiments wherein said immune suppressive factors are FGF-2.

Further methods include embodiments wherein said immune suppressive factors are PGE-2.

Further methods include embodiments wherein said immune suppressive factors are soluble HLA-G.

Further methods include embodiments wherein said tumor associated fibroblasts inhibit and/or kill immune system cells.

Further methods include embodiments wherein said immune system cells are T cells.

Further methods include embodiments wherein said T cells are CD4 T cells.

Further methods include embodiments wherein said T cells are CD4 T cells capable of secreting more interferon gamma as compared to interleukin-4.

Further methods include embodiments wherein said T cells are Th1 cells.

Further methods include embodiments wherein said T cells are CD8 T cells.

Further methods include embodiments wherein said T cells are cytotoxic T cells.

### BRIEF DESCRIPTION OF THE FIGURES

FIG. 1 is a bar graph showing cell metastases based on different concentrations of QUADRAMUNE™

FIG. 2 is a bar graph showing cell metastases based on QUADRAMUNE™ a vaccine, and a combination thereof.

FIG. 3 is a bar graph showing mesenchymals stem cell levels (as a percentage of splenocytes) based on administration of QUADRAMUNE™ and a vaccine.

### DETAILED DESCRIPTION OF THE INVENTION

The invention discloses use of QuadraMune™, a composition of pterostilbene, Nigella sativa extract, green tea extract, and broccoli for reduction in cancer, and specifically, suppression of myeloid suppressor cell number and activity.

A composition in accordance with the present invention containing pterostilbene, thymoquinone, EGCG, and sulforaphane, or a pharmaceutically acceptable salt thereof, can be prepared by conventional procedures for blending and mixing compounds. Preferably, the composition also includes an excipient, most preferably a pharmaceutical excipient. Compositions containing an excipient and incorporating the pterostilbene can be prepared by procedures known in the art. For example, the ingredients can be formulated into tablets, capsules, powders, suspensions, solutions for oral administration and solutions for parenteral administration including intravenous, intradermal, intramuscular, and subcutaneous administration, and into solutions for application onto patches for transdermal application with common and conventional carriers, binders, diluents, and excipients.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

We disclose that the invention further provides nutraceutical compositions comprising the chemical compound of the invention, or a pharmaceutically acceptable salt or derivative thereof, together with one or more nutraceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof. An oral composition can generally include an inert diluent or an edible carrier. The nutraceutical composition can comprise a functional food component or a nutrient component. The term “functional food” refers to a food which contains one or a combination of components which affects functions in the body so as to have positive cellular or physiological effects. The term “nutrient” refers to any substance that furnishes nourishment to an animal.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents.

Lotions may be formulated with an aqueous or oily base and will in general also contain one or more emulsifying agents, stabilizing agents, dispersing agents, suspending agents, thickening agents, or coloring agents.

Compositions suitable for topical administration in the mouth include lozenges comprising the active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules and lozenges for oral administration and liquids for intravenous administration and continuous infusion are preferred compositions. Solutions or suspensions for application to the nasal cavity or to the respiratory tract are preferred compositions. Transdermal patches for topical administration to the epidermis are preferred.

Details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED.sub.50 and LD.sub.50, may be determined by standard pharmacological procedures in cell cultures or experimental animals. The dose ratio between therapeutic and toxic effects is the therapeutic index and may be expressed by the ratio LD.sub.50/ED.sub.50. Pharmaceutical compositions exhibiting large therapeutic indexes are preferred.

The dosage of compound used in accordance with the invention varies depending on the compound and the condition being treated. The age, lean body weight, total weight, body surface area, and clinical condition of the recipient patient; and the experience and judgment of the clinician or practitioner administering the therapy are among the factors affecting the selected dosage. Other factors include the route of administration, the patient's medical history, the severity of the disease process, and the potency of the particular compound. The dose should be sufficient to ameliorate symptoms or signs of the disease treated without producing unacceptable toxicity to the patient. The dosage may be varied by titration of the dosage to the particular circumstances of this invention to produce the desired therapeutic effect.

### Example 1: Reduction in Lung Metastasis by QUADRAMUNE™

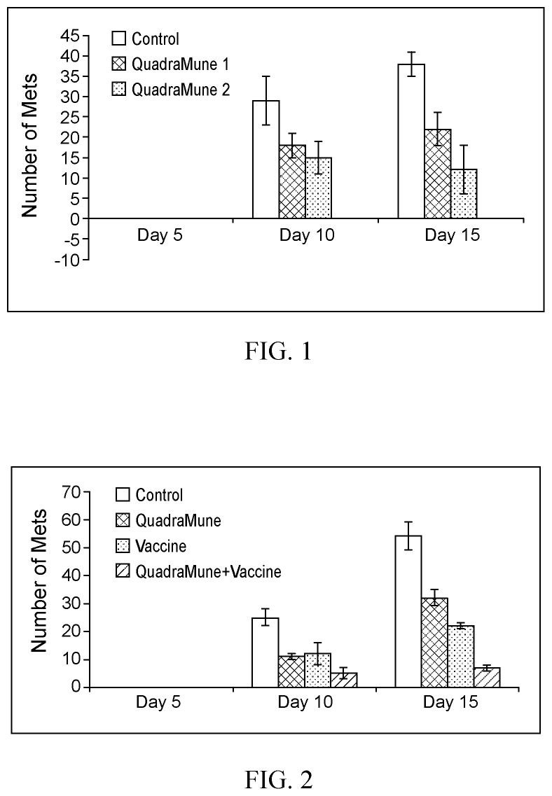

Female BALB/c mice were inoculated intravenously with 500,000 4T1 cells. QUADRAMUNE™ was administered daily by gavage at a concentration 1 (100 ug of broccoli sprout extract, Nigella sativa, and green tea extract, and 50 ug of pterostilbene), and concentration 2 (200 ug of broccoli sprout extract, Nigella sativa, and green tea extract, and 100 ug of pterostilbene). Metastasis were measured at the indicated timepoints with H and E staining. Results are shown in FIG. 1.

### Example 2: QUADRAMUNE™ Enhances Cancer Vaccine Efficacy

Female BALB/c mice were inoculated intravenously with 500,000 4T1 cells. QUADRAMUNE™ was administered daily by gavage at a concentration 1 (100 ug of broccoli sprout extract, Nigella sativa, and green tea extract, and 50 ug of pterostilbene). Vaccine comprised administration subcutaneously of 1 million lyzed 4T1 cells administered as described. Metastasis were measured at the indicated timepoints with H and E staining. Results are shown in FIG. 2.

### Example 3: QUADRAMUNE™ Decreases MSC

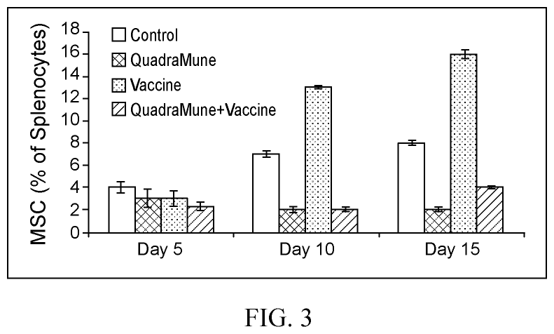

Female BALB/c mice were inoculated intravenously with 500,000 4T1 cells. QUADRAMUNE™ was administered daily by gavage at a concentration 1 (100 ug of broccoli sprout extract, Nigella sativa, and green tea extract, and 50 ug of pterostilbene). Vaccine comprised administration subcutaneously of 1 million lyzed 4T1 cells administered as described². MSC were assessed by flow cytometry for cells staining with CD11b and Gr1. Cells are expressed as percentage of splenocytes. Results are shown in FIG. 3.

1. A method of inhibiting number and/or activity of myeloid suppressor cells comprising: administering to myeloid suppressor cells a therapeutic combination comprising: a) Green Tea and/or extract thereof; b) Blueberry and/or extract thereof; c) Nigella sativa and/or extract thereof; and d) broccoli and/or extract thereof.

2. The method of claim 1, wherein said green tea extract is epigallocatechin-3-gallate or an analogue thereof.

3. The method of claim 1, wherein said blueberry extract is pterostilebene or an analogue thereof.

4. The method of claim 1, wherein said Nigella sativa extract is thymoquinone or an analogue thereof.

5. The method of claim 1, wherein said broccoli extract is sulforaphane or an analogue thereof.

6. The method of claim 1, wherein said therapeutic combination is administered at a dosage and frequency sufficient to inhibit MSC number and/or activity.

7. The method of claim 6, wherein inhibition of MSC number and/or activity in the host is associated with enhancement of natural killer cell activity.

8. The method of claim 7, wherein said natural killer cell activity is quantified by ability to lyse a virally infected cell.

9. The method of claim 7, wherein said natural killer cell activity is quantified by ability to lyse K562 cells.

10. The method of claim 7, wherein said natural killer cell activity is quantified by ability to lyse YAC-1 cells.

11. The method of claim 6, wherein inhibition of MSC number and/or activity in the host is associated with enhancement of interferon production.

12. The method of claim 6, wherein inhibition of MSC number and/or activity in the host is accomplished by enhancement of T cell activation.

13. The method of claim 12, wherein said T cell activation is induction of T helper cell 1 activity.

14. The method of claim 13, wherein said T helper cell 1 activity comprises production of interferon gamma.

15. The method of claim 12, wherein said T cell activation is induction of T cytotoxic cell activity.

16. The method of claim 1, wherein said therapeutic combination is administered at a dosage and frequency sufficient to suppress growth of a tumor.

17. The method of claim 16, wherein said tumor growth in the host is associated with suppression of cancer angiogenesis.

18. The method of claim 17, wherein said cancer angiogenesis comprises of: a) endothelial cell detachment; b) migration towards a chemotactic gradient; and c) tube formation.

19. The method of claim 1, wherein said therapeutic mixture decreases tumor associated fibroblasts.

20. The method of claim 19, wherein said tumor associated fibroblasts secrete immune suppressive factors.

21-39. (canceled)
